(19) United States
(12) Patent Application Publication
(10) Pub. No.: US 2010/0298256 A1
(43) Pub. Date: Nov. 25, 2010

(54) ANTIVIRAL COMPOUNDS

(76) Inventors: Steven Dong, San Francisco, CA (US); Mel C. Schroeder, Dexter, MI (US)

Correspondence Address: EcoTech Law Group, P.C., 201 SPEAR STREET, SUITE 1100, SAN FRANCISCO, CA, 94105, US

(21) Appl. No.: 12/810,147

(22) PCT Filed: Dec. 23, 2008
(86) PCT No.: PCT/US08/14015

§ 371 Date: Aug. 3, 2010

Related U.S. Application Data
(60) Provisional application No. 61/017,116, filed on Dec. 27, 2007.

(51) Int. Cl. A61K 31/7068; C07H 19/14; C07H 19/056; C07H 19/10; C07F 9/6512; A61K 31/7056; A61K 31/675; A61P 31/12; A61P 31/16; A61P 31/14

(52) U.S. Cl. 514/49; 536/26.7; 536/26.9; 536/26.8; 544/243; 544/244; 514/52; 514/81; 514/86; 514/80

(57) ABSTRACT

Lipid-modified phosphodiester nucleoside prodrugs are described herein. The prodrugs can be used to treat viral infections and cancer.

### CROSS-REFERENCE TO RELATED APPLICATIONS

The present application claims the benefit under 35 U.S.C. §119(e)(1) of U.S. provisional application Ser. No. 61/017,116, filed Dec. 27, 2007, which application is incorporated herein by reference in its entirety.

### BACKGROUND OF THE INVENTION

1. Field of the Invention

The present invention relates to novel antiviral compounds, methods of manufacture of said compounds, and methods of using said compounds to treat a variety of medical disorders, including, for example, viral infections and cancer.

2. Background of the Invention

A nucleoside is composed of a nucleobase attached to a ribose or deoxyribose ring. Nucleoside analogs in which the ribose is replaced with a modified ring nucleus (“cyclic”) or with a non-ring nucleus (“acyclic”) have been described. A subset of these cyclic and acyclic nucleoside analogs has shown antiviral activity, and some are used clinically to treat a number of viral infections. Examples of cyclic nucleoside analogs include brivudine, zidovudine (AZT, Retrovir), didanosine (ddl, Videx), zalcitabine (ddC, Hivid), stavudine (d4T, Zerit), and abacavir (Ziagen). Examples of acyclic nucleoside analogs include acyclovir (Zovirax), penciclovir (Denavir), omaciclovir (H2G), and ganciclovir (Cytovene). Some antiviral nucleoside analogs are phosphorylated up to three times intracellularly by kinases to produce the nucleoside analog tri-phosphate. These phosphorylated nucleoside analogs exert their antiviral activity by a variety of mechanisms of action, including inhibition of viral enzymes such as DNA polymerase and reverse transcriptase.

Ribavirin is an example of a cyclic nucleoside analog that shows some antiviral activity against RNA and DNA viruses such as hepatitis C virus (HCV). Unlike other nucleoside analogs, the predominant mechanism(s) of ribavirin action against viruses such as HCV are yet to be established. [Dixit, N M; Perelson, A S Cell Mol Life Sci 2006, 63, 832; incorporated herein by reference]. However, the active form of ribavirin is comprised of its three 5′-phosphorylated states [Wu, J Z; Larson, G; Walker, H; Shim, J H; Hong, Z Antimicro Agent Chemother 2005, 49, 2164; incorporated herein by reference]. For example, ribavirin 5′-monophosphate can inhibit inosine monophosphate dehydrogenase (IMPDH), an enzyme which plays a role in supporting viral replication [Gish, R G Antimicrob Chemother 2005, 57, 8; incorporated herein by reference].

A major limitation of directly administering phosphorylated compounds, such as phosphorylated nucleoside analogs, is that they are poorly absorbed from the GI tract. Additionally many must be parenterally administered. Furthermore, the negatively charged phosphate moiety can interfere with cellular penetration, resulting in reduced antiviral or antiproliferative activity.

In some cases, phosphorylated nucleoside analogs are also associated with toxic effects. For example, one of the chief limitations of ribavirin is the side effect of hemolytic anemia [Russmann, S; Grattagliano, I; Portincasa, P; Palmieri, V O; Palasciano, G Curr Med Chem 2006, 13, 3351; incorporated herein by reference]. The anemia has been attributed to the excessive build-up of ribavirin-5′-tri-phosphate (RTP) in erythrocytes which can competitively inhibit adenosine tri-phosphate (ATP) dependent utilization. Erythrocytes accumulate RTP because they lack dephosphorylating enzymes that can degrade RTP back to ribavirin.

Therefore, there is a continuing need for less toxic, more effective phosphorylated nucleoside analogs to treat a variety of disorders, such as those caused by viral infection, cancer, and other diseases relating to inappropriate cell proliferation, e.g., autoimmune diseases.

### SUMMARY OF THE INVENTION

The present invention provides a means of delivering phosphorylated nucleoside analogs to virally infected cells or cancer cells by providing lipid-modified phosphodiester nucleoside prodrugs as antiviral agents. These lipid-modified phosphodiester nucleoside prodrugs minimize deleterious side effects over the parent nucleoside analog when administered to a subject in need thereof.

In a first aspect, the present invention provides a lipid-modified phosphodiester nucleoside prodrug compound and pharmaceutical compositions thereof. This composition, in some embodiments, is a phosphorylated nucleoside analog covalently linked (directly or indirectly through a linker molecule) to a substituted lipid such as unsubstituted alkylglycerol, alkylpropanediol, or alkylethanediol that acts as a prodrug of an antiviral agent. This composition, in other embodiments, is useful in the prevention and/or treatment of viral infections, especially hepatitis C (HCV) in adults with detectable HCV and compensated liver disease; diseases such as respiratory syncytial virus (RSV), influenza, and SARS; diseases such as genital herpes (HSV-1/2), shingles (VZV), mononucleosis (EBV), CMV retinitis, and/or other herpes virus infections stemming from HHV-6A, HHV-6B, and HHV-8; and for other diseases and conditions that benefit from antiviral drug treatment.

In a second aspect, the present invention provides a method of preparing lipid-modified phosphodiester nucleoside prodrug compounds. This method, in some embodiments, utilizes phosphoramidite chemistry to synthesize the compounds of this invention.

In a third aspect, the present invention provides therapeutic methods and compositions for use in those methods in which a patient is administered a therapeutically effective amount of (a) a lipid-modified phosphodiester nucleoside prodrug of this invention; and optionally, (b) a pharmaceutically compatible carrier or diluent, for the treatment of viral infections. In some embodiments, the present invention provides therapeutic methods and compositions for use in those methods in which a patient is co-administered (a) a lipid-modified phosphodiester nucleoside prodrug of this invention; (b) one or more additional antiviral therapeutics; and optionally, (c) a pharmaceutically compatible carrier or diluent, for the treatment of viral infections. In some embodiments, a lipid-modified phosphodiester nucleoside prodrug of this invention and additional antiviral therapeutic(s) are administered separately. In other embodiments, one, two, or three agents are optionally admixed with a carrier.

Non-limiting examples of such co-administered additional antiviral therapeutics include: (a) interferons such as peginterferon alfa-2a, pegylated interferon alfa-2b, interferon alfa-2b, interferon alfa-2a, and consensus intereferon; (b) HCV protease inhibitors such as telaprevir and boceprevir; (c) HCV polymerase inhibitors such as valopcitabine and R-1626; (d) neuramindase inhibitors such as zanamivir and oseltamivir; and (e) M2 channel blockers such as amantadine and rimantadine.

### BRIEF DESCRIPTION OF THE FIGURES

FIG. 1 shows a representative method of preparing a lipid-modified phosphodiester nucleoside prodrug utilizing the nucleoside analog ribavirin.

FIG. 2 shows the Pharmacokinetics of (ODE) phosphodiester ribavirin prodrug in mouse plasma following intravenous administration of 5 mg/kg and oral administration of 30 mg/kg in male mice (N=3).

### DETAILED DESCRIPTION OF THE INVENTION

The detailed description of the different aspects and embodiments of the present invention is organized at follows: Section I provides useful definitions; Section II describes the compounds of the present invention and methods of preparation; Section III provides methods of treatment, administration, formulation, and describes unit dose form for the present invention; and Section IV provides illustrative methods for synthesizing and demonstrating the activity of the compounds of the present invention. This detailed description is organized into sections only for the convenience of the reader, and disclosure found in any section is applicable to disclosure elsewhere herein.

### SECTION I

### Definitions

As used herein, the term “alkyl” refers to a monovalent straight or branched chain or cyclic radical of from one to twenty-four (C₁-C₂₄), carbon atoms, including methyl, ethyl, n-propyl, isopropyl, n-butyl, isobutyl, tert-butyl, n-hexyl, and the like.

As used herein, “substituted alkyl” comprises alkyl groups further bearing one or more substituents selected from hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, trifluoromethyl, cyano, nitro, nitrone, amino, amido, formyl, acyl, oxyacyl, carboxyl, carbamate, sulfonyl, sulfonamide, sulfuryl, and the like.

As used herein, “alkenyl” refers to straight or branched chain hydrocarbyl groups having one or more carbon-carbon double bonds, and having in the range of about 2 up to 24 (C₁-C₂₄) carbon atoms, and “substituted alkenyl” refers to alkenyl groups further bearing one or more substituents as defined under substituted alkyl.

As used herein, “aryl” refers to aromatic groups having in the range of 6 up to 14 carbon atoms and “substituted aryl” refers to aryl groups further bearing one or more substituents as defined under substituted alkyl.

As used herein, “heteroaryl” refers to aromatic groups containing one or more heteroatoms (e.g., N, O, S, or the like) as part of the ring structure, and having in the range of 3 up to 14 carbon atoms, and “substituted heteroaryl” refers to heteroaryl groups further bearing one or more substituents as defined under substituted alkyl.

As used herein, the term “bond” or “valence bond” refers to a linkage between atoms consisting of an electron pair.

As used herein, the term “pharmaceutically acceptable salts” refers to both acid and base addition salts that can be used in preparations intended for pharmaceutical use.

As used herein, the term “prodrug” refers to analogs, derivatives, or variants of pharmaceutically active compounds that differ from the corresponding pharmaceutically active compound by having chemically or metabolically cleavable or lacking addable groups that become the pharmaceutically active compound by solvolysis or other enzymatic action under in vivo physiological conditions. Prodrugs maybe much less active than the “parent” compound in such vivo physiological conditions.

As used herein, the term “lipid” refers to a chain comprised either individually or in combination with alkyl, substituted alkyl, alkenyl, substituted alkenyl, aryl, heteroaryl, groups and the like as defined above. Lipids, for purposes of the present invention, include fatty acids, neutral fats, waxes, steroids and other illustrative lipids.

As used herein, the term “phosphodiester” refers to a group containing a phosphorus atom in a phosphate group that is bonded via two ester bonds to two other alkyl groups or combinations of such groups comprised either individually of or in combination with alkyl, substituted alkyl, alkenyl, aryl, heteroaryl, lipid, nucleoside groups and the like as defined above.

As used herein, the term “acyclic” denotes the absence of a cyclic structure within the nucleus of the nucleoside analog. As used herein, the term “cyclic” refers denotes the presence of a cyclic structure within the nucleus of the nucleoside analog. As used herein, the term “modified ring” refers to the presence of a structurally modified ribose within the nucleus of the nucleoside analog.

The terms “co-administration” and “co-administering”, as used herein, refer to the administration of a substance before, concurrently, or after the administration of another substance, such that the biological effects of the substances overlap and are experienced at least in part concurrently by the subject to which they are administered. In some embodiments the combination agent that includes a lipid-modified phosphodiester nucleoside prodrug of this invention and other therapeutic agents are administered immediately before, concurrently with or immediately after the administration of each dose of the therapeutic agents. In other embodiments, the agents are admixed together prior to administration to the patient. In other embodiments, the agents are co-administered by different methods of administration. In other embodiments, the therapeutic agent is administered immediately before, concurrently with or immediately after the administration of a dose of the lipid-modified phosphodiester nucleoside prodrugs of this invention and the remaining daily doses of lipid-modified phosphodiester nucleoside prodrugs are administered alone without the therapeutic agent, i.e. in the absence of the therapeutic agent.

As used herein, the term “parenteral” refers to subcutaneous, intravenous, intra-arterial, intramuscular or intravitreal injection or infusion techniques.

### SECTION II

The lipid-modified phosphodiester nucleoside prodrug compounds of the invention have the structure:

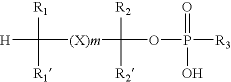

R₁ and R₁′ are independently —H, substituted and unsubstituted —O(C₁-C₂₄)alkyl, —O(C₁-C₂₄)alkenyl, —O(C₁-C₂₄)acyl, —S(C₁-C₂₄)alkyl, —S(C₁-C₂₄)alkenyl, or —S(C₁-C₂₄)acyl, wherein at least one of R₁ and R₁′ is not —H, and wherein said alkenyl or acyl moieties optionally have 1 to 6 double bonds;

R₂ and R₂′ are independently —H, substituted and unsubstituted —O(C₁-C₇)alkyl, —O(C₁-C₇)alkenyl, —S(C₁-C₇)alkyl, —S(C₁-C₇)alkenyl, —O(C₁-C₇)acyl, —S(C₁-C₇)acyl, —N(C₁-C₇)acyl, —NH(C₁-C₇)alkyl, —N((C₁-C₇)alkyl)₂, oxo, halogen, —NH₂, —OH, or —SH;

R₃ is a pharmaceutically active nucleoside including acyclic or cyclic analogs having a ribose or a modified ring or a non-ring structure, in each case having a modified structure containing at least one modifiable hydroxyl group in which the ribose nucleoside is replaced with a modified ring (“cyclic”) or with a non-ring structure (“acyclic”). Examples of cyclic nucleoside analogs include ribavirin (Copegus, Rebetol, Ribasphere), viramidine (Taribavirin), valopicitabine (NM283), NM107, MK608, R1479, brivudine, zidovudine (AZT, Retrovir), didanosine (ddl, Videx), zalcitabine (ddC, Hivid), stavudine (d4T, Zerit), and abacavir (Ziagen), idoxuridine, lobucavir, cyclopropavir, lamivudine, cyclohexenyl G, and maribavir. Examples of acyclic nucleoside analogs include acyclovir (Zovirax), penciclovir (Denavir), omaciclovir (H2G), S2242, A-5021, and ganciclovir (Cytovene).

X, when m is greater than 0, is:

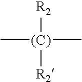

and m is an integer from 0 to 6.

In some embodiments, m=0, 1 or 2, and R₂ and R₂′ are H. The corresponding analogs can then be described as ethanediol, propanediol or butanediol derivatives of the lipid-modified phosphodiester nucleoside prodrug compounds of the invention. In one embodiment, the derivative has the structure:

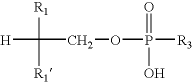

wherein R₁ and R₁′ and R₃ are as defined above.

In some embodiments, the derivative has the structure:

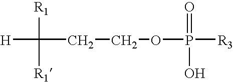

wherein m=1 and R₁, R₁′, and R₃ are as defined above.

Similarly, in other embodiments, the invention provides glycerol derivatives having the structure:

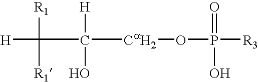

wherein m=1, R₂═H, R₂¹=0H, and R₂ and R₂′ on C^{α} are both —H. In compounds of the invention having a glycerol residue, the —P(O)OH—R₃ moiety may be joined at either the sn-3 or sn-1 position of glycerol.

In some embodiments of the lipid-modified phosphodiester nucleoside prodrug compounds of this invention, R₁ is an alkoxy group having the formula —O—(CH₂)t-CH₃, wherein t is 0-24. In another embodiment, t is 11-19. In another embodiment t is 15 or 17.

Certain compounds of the invention possess one or more chiral centers, e.g., in the sugar moieties, and may thus exist in optically active forms. Likewise, when the compounds contain an alkenyl group or an unsaturated alkyl or acyl moiety there exists the possibility of cis- and trans-isomeric forms of the compounds. Additional asymmetric carbon atoms can be present in a substituent group such as an alkyl group. The R- and S-isomers and mixtures thereof, including racemic mixtures as well as mixtures of cis- and trans-isomers are provided by this invention. All such isomers as well as mixtures thereof are provided in the invention. If a particular stereoisomer is desired, it can be prepared by methods well known in the art for other compounds by using stereospecific reactions with starting materials that contain the asymmetric centers and are already resolved or, alternatively, by methods that lead to mixtures of the stereoisomers, followed by resolution by known methods.

### Method of Preparation of Lipid-Modified Phosphodiester Nucleoside Prodrugs

In one aspect, the present invention provides lipid-modified phosphodiester nucleoside prodrugs in which a nucleoside hydroxyl group is covalently linked (directly or indirectly through a linker molecule) to a substituted or unsubstituted alkylglycerol, alkylpropanediol, alkylethanediol, or related moiety to yield the phosphodiester. In one embodiment, the lipid-modifying group is octadecyl-ethanediol (“ODE”). Table 1 illustrates non-limiting examples of such lipid-modified phosphodiester nucleoside prodrugs provided by the invention.

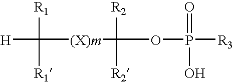

| Compound | R₁ | R_{1′} | X | m | R₂ | R_{2′} | R₃ |
|---|---|---|---|---|---|---|---|
| ODE-phospho- ribavirin | CH₃(CH₂)₁₇O | H | CH₂ | 0 | H | H | 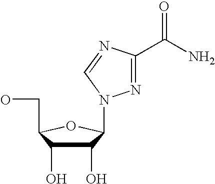 |
| ODE-phospho- viramidine | CH₃(CH₂)₁₇O | H | CH₂ | 0 | H | H | 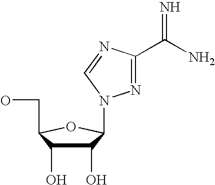 |
| ODE-phospho- NM107 | CH₃(CH₂)₁₇O | H | CH₂ | 0 | H | H | 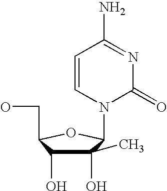 |
| ODE-phospho- valopicitabine | CH₃(CH₂)₁₇O | H | CH₂ | 0 | H | H | 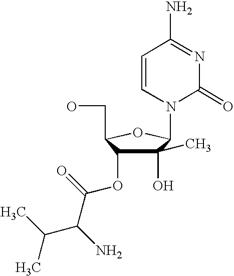 |
| ODE-phospho- MK608 | CH₃(CH₂)₁₇O | H | CH₂ | 0 | H | H | 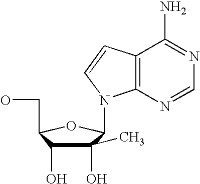 |
| ODE-phospho- R1479 | CH₃(CH₂)₁₇O | H | CH₂ | 0 | H | H | 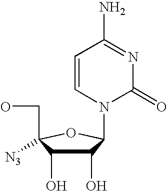 |
| ODE-phospho- brivudine | CH₃(CH₂)₁₇O | H | CH₂ | 0 | H | H | 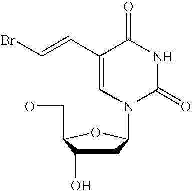 |
| ODE-phospho- zidovudine (azt) | CH₃(CH₂)₁₇O | H | CH₂ | 0 | H | H | 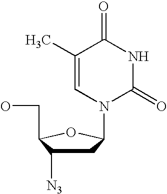 |
| ODE-phospho- didanosine (ddI) | CH₃(CH₂)₁₇O | H | CH₂ | 0 | H | H | 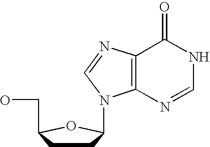 |
| ODE-phospho- zalcitabine (ddC) | CH₃(CH₂)₁₇O | H | CH₂ | 0 | H | H | 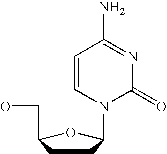 |
| ODE-phospho- idoxuridine | CH₃(CH₂)₁₇O | H | CH₂ | 0 | H | H | 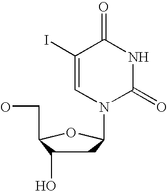 |
| ODE-phospho- lobucavir | CH₃(CH₂)₁₇O | H | CH₂ | 0 | H | H | 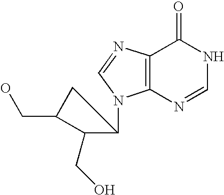 |
| ODE-phospho- cyclopropavir | CH₃(CH₂)₁₇O | H | CH₂ | 0 | H | H | 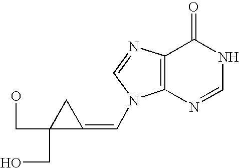 |
| ODE-phospho- lamivudine | CH₃(CH₂)₁₇O | H | CH₂ | 0 | H | H | 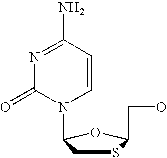 |
| ODE-phospho- stavudine | CH₃(CH₂)₁₇O | H | CH₂ | 0 | H | H | 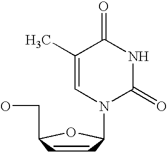 |
| ODE-phospho- abacavir | CH₃(CH₂)₁₇O | H | CH₂ | 0 | H | H | 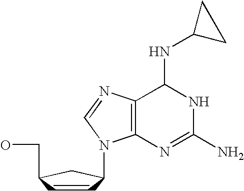 |
| ODE-phospho- cyclohexenyl G | CH₃(CH₂)₁₇O | H | CH₂ | 0 | H | H | 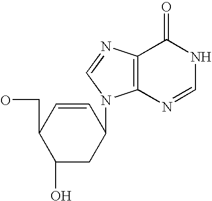 |
| ODE-phospho- maribavir | CH₃(CH₂)₁₇O | H | CH₂ | 0 | H | H | 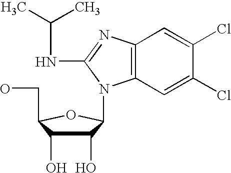 |
| ODE-phospho- acyclovir | CH₃(CH₂)₁₇O | H | CH₂ | 0 | H | H | 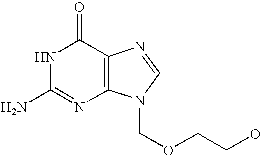 |
| ODE-phospho- penciclovir | CH₃(CH₂)₁₇O | H | CH₂ | 0 | H | H | 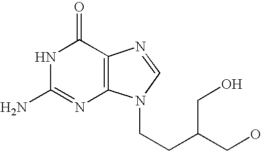 |
| ODE-phospho- omaciclovir | CH₃(CH₂)₁₇O | H | CH₂ | 0 | H | H | 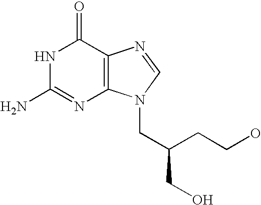 |
| ODE-phospho- S2242 | CH₃(CH₂)₁₇O | H | CH₂ | 0 | H | H | 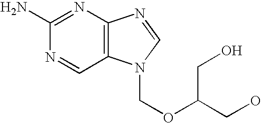 |
| ODE-phospho- A-5021 | CH₃(CH₂)₁₇O | H | CH₂ | 0 | H | H | 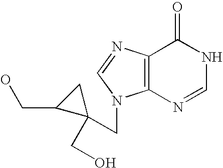 |
| ODE-phospho- ganciclovir | CH₃(CH₂)₁₇O | H | CH₂ | 0 | H | H | 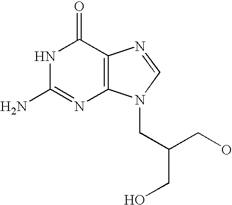 |

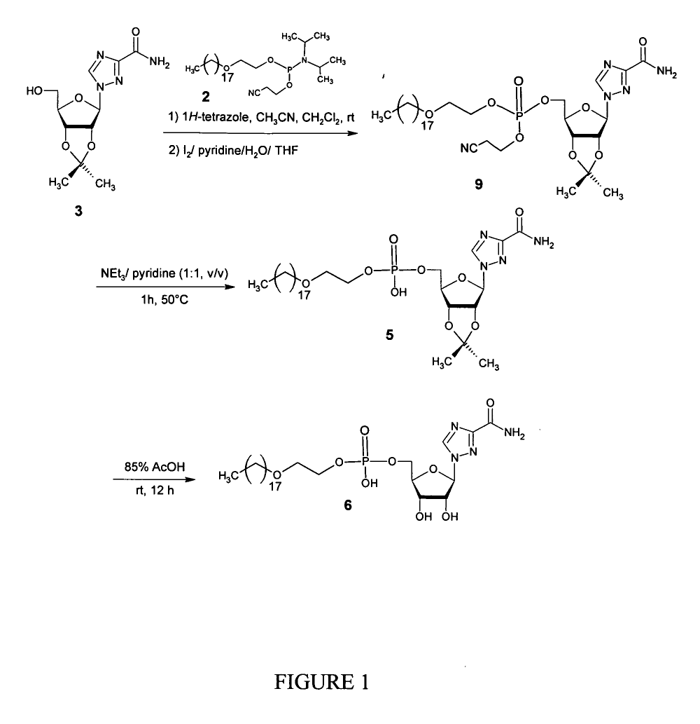

In some embodiments, the present invention provides a general method of preparing lipid-modified phosphodiester nucleoside prodrugs which utilizes phosphoramidite chemistry. A representative example utilizing the nucleoside analog ribavirin is shown in FIG. 1. In one aspect, an appropriately protected cyclic and acyclic nucleoside, such as 2′,3′-acetonide protected ribavirin 3, is coupled with a lipid-modified phosphoramidite such as 1-O-octadecyl-ethanediol-2-(2-cyanoethyl-N,N-diisopropyl)-phosphoramidite 2. Subsequent oxidation with an oxidant such as I₂ provides the lipid-modified phosphotriester nucleoside analog, such as 9. Base-mediated removal of the cyanoethoxy group from the phosphotriester provides the phosphodiester, such as 5. If required, appropriate deprotection of the nucleoside, such as removal of the acetonide protecting group from 5, provides the final lipid-modified phosphodiester nucleoside prodrugs described in this invention, such as the octadecyl-ethanediol-modified (ODE) phosphodiester ribavirin prodrug 6.

### SECTION III

### Methods of Treating Disease

This invention provides methods of treating or preventing disorders related to disease, viral infections and cancer, and the like. The methods comprise administering to a human or other mammal in need thereof a therapeutically effective amount of the lipid-modified phosphodiester nucleoside prodrugs of this invention.

With respect to disorders associated with viral infections or inappropriate cell proliferation, e.g., cancer, the “therapeutically effective amount” is determined with reference to the recommended dosages of the antiviral or anticancer parent compound. The selected dosage will vary depending on the activity of the selected compound, the route of administration, the severity of the condition being treated, and the condition and prior medical history of the patient being treated. However, it is within the scope of the skilled artisan to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient will depend on a variety of factors, including the body weight, general health, diet, time, and route of administration and combination with other drugs, and the severity of the disease being treated.

Generally, the compounds of the present invention are dispensed in unit dosage form comprising 1% to 100% of active ingredient. The range of therapeutic dosage is from about 0.01 to about 1,000 mg/kg (of patient weight)/day, for example, from about 0.10 mg/kg/day to 100 mg/kg/day being preferred, when administered to patients, e.g., humans, as a drug. Actual dosage levels of active ingredients in the pharmaceutical compositions of this invention may be varied so as to administer an amount of the active compound(s) that is effective to achieve the desired therapeutic response for a particular patient.

In some embodiments, the present invention provides a method of treatment of virus infections, including infections caused by RNA and DNA viruses, said method comprising administering to a human or other mammal in need thereof a therapeutically effective amount of lipid-modified phosphodiester nucleoside prodrugs of the invention. Illustrative examples of the use of lipid-modified phosphodiester nucleoside prodrugs and dosage unit forms, method of administration, and dosage schedule are listed in Table 2.

TABLE 2
Method of administration and dosages for administration of lipid-modified
phosphodiester nucleoside prodrugs as single agents.

| | | | Dose amount & Admin Method & |
|---|---|---|---|
| Combo | Indication | API | Schedule |
| | | RNA Viruses | |
| 1. | Hepatitis C | ODE-Phosphodiester Ribavirin | 1200 mg po, 48 weeks |
| | | Prodrug | |
| 2. | RSV | ODE-Phosphodiester Ribavirin | 1g q6h, 4d, then 500 mg q8h for 3d |
| | | Prodrug | po |
| 3. | Influenza | ODE-Phosphodiester Ribavirin | 1g q6h, 4d, then 500 mg q8h for 3d |
| | | Prodrug | po |
| 4. | SARS | ODE-Phosphodiester Ribavirin | 1g q6h, 4d, then 500 mg q8h for 3d |
| | | Prodrug | po |
| 5. | Lassa fever | ODE-Phosphodiester Ribavirin | 1g q6h, 4d, then 500 mg q8h for 3d |
| | | Prodrug | po |
| | | DNA viruses | |
| 6. | Genital herpes | ODE-Phosphodiester Acyclovir | 1000 mg BID po for 10 d |
| | HSV-1/2 | Prodrug | |
| 7. | Shingles VZV, | ODE-Phosphodiester Omaciclovir | 1000 mg QD po for 7 d |
| | HHV-3 | Prodrug | |
| 8. | Mononucleosis | ODE-Phosphodiester Omaciclovir | 2000 mg BID po for 3 weeks |
| | EBV, HHV-4 | Prodrug | |
| 9. | CMV retinitis | ODE-Phosphodiester Ganciclovir | 900 mg BID po for 3 weeks |
| | HHV-5 | Prodrug | |
| 10. | HHV-6A | ODE-Phosphodiester A-5021 Prodrug | 1000 mg QD po for 6 mo |
| 11. | HHV-6B | ODE-Phosphodiester A-5021 Prodrug | 1000 mg QD po for 6 mo |
| 12. | HHV-8 | ODE-Phosphodiester Ganciclovir | 900 mg BID po for 3 weeks |
| | | Prodrug | |

In some embodiments, this invention provides a method of treatment of hepatitis C (HCV) employing octadecyl-ethanediol-modified (ODE) phosphodiester ribavirin prodrugs, said method comprising administering to a human or other mammal in need there of a therapeutically effective amount of the octadecyl-ethanediol-modified (ODE) phosphodiester ribavirin prodrugs of the invention. In other embodiments, octadecyl-ethanediol-modified (ODE) phosphodiester ribavirin prodrug is administered orally (po) in a dosage unit of about 100 mg to 4000 mg per day once a day (qd) for 48 weeks to adults with detectable hepatitis C virus and compensated liver disease. The actual dose administered varies depending on a number of patient factors including patient weight.

In some embodiments, this invention provides a method of treatment of respiratory syncytial virus (RSV) employing octadecyl-ethanediol-modified (ODE) phosphodiester ribavirin prodrug, said method comprising administering to a human or other mammal in need there of a therapeutically effective amount of the octadecyl-ethanediol-modified (ODE) phosphodiester ribavirin prodrugs of the invention. In other embodiments, octadecyl-ethanediol-modified (ODE) phosphodiester ribavirin prodrug is administered orally (po) in a dosage unit of about 100 mg to 4000 mg every 6 hours (q6 h) for 4 days, then in a dosage unit of about 100 mg to 4000 mg every 8 hours (q8 h) for 3 days to children with detectable RSV infection and severe bronchiolitis and/or pneumonia. The actual dose administered varies depending on a number of patient factors including patient weight.

In some embodiments, this invention provides a method of treatment of influenza employing octadecyl-ethanediol-modified (ODE) phosphodiester ribavirin prodrug, said method comprising administering to a human or other mammal in need there of a therapeutically effective amount of the octadecyl-ethanediol-modified (ODE) phosphodiester ribavirin prodrugs of the invention. In other embodiments, octadecyl-ethanediol-modified (ODE) phosphodiester ribavirin prodrug is administered orally (po) in a dosage unit of about 100 mg to 4000 mg every 6 hours (q6 h) for 4 days, then in a dosage unit of about 100 mg to 4000 mg every 8 hours (q8 h) for 3 days to adults for the treatment of uncomplicated acute illness due to influenza infection. Symptoms of influenza may include a fever >100° F.; respiratory symptoms such as cough, nasal symptoms, or sore throat; and systemic symptoms such as myalgia, chill/swats, malaise, fatigue or headache. The actual dose administered varies depending on a number of patient factors including patient weight.

In some embodiments, this invention provides a method of treatment of severe acute respiratory syndrome (SARS) employing octadecyl-ethanediol-modified (ODE) phosphodiester ribavirin prodrug, said method comprising administering to a human or other mammal in need there of a therapeutically effective amount of the octadecyl-ethanediol-modified (ODE) phosphodiester ribavirin prodrugs of the invention. In other embodiments, octadecyl-ethanediol-modified (ODE) phosphodiester ribavirin prodrug is administered orally (po) in a dosage unit of about 100 mg to 4000 mg every 6 hours (q6 h) for 4 days, then in a dosage unit of about 100 mg to 4000 mg every 8 hours (q8 h) for 3 days to adults with detectable SARS infection and/or SARS symptoms such as a fever ≧100.4° F.; positive chest x-ray findings of atypical pneumonia or respiratory distress syndrome; contact (sexual or casual) with someone with a diagnosis of SARS within the last 10 days; and/or travel to any of the regions identified by the WHO as areas with recent local transmission of SARS. The actual dose administered varies depending on a number of patient factors including patient weight.

In some embodiments, the present invention provides lipid-modified phosphodiester nucleoside prodrugs useful for the treatment of disorders caused by other viral infections. Indications appropriate to such treatment include susceptible viruses such as hepatitis B virus, human immunodeficiency virus (HIV), herpes simplex virus-1 (HSV-1), herpes simplex virus-2 (HSV-2), varicella zoster virus (VZV, HHV-3), Epstein-Barr virus (EBV, HHV-4) cytomegalovirus (CMV, HHV-5), human herpes virus 6A (HHV-6A), human herpes virus 6B (HHV-6B), Kaposi's Sarcoma Associated Virus (KSHV, HHV-8), and diseases caused by orthopox viruses (e.g., variola major and minor, vaccinia, smallpox, cowpox, camelpox, monkeypox, and the like), ebola virus, papilloma virus, and the like.

In some embodiments, there are provided methods for treating disorders caused by inappropriate cell proliferation, e.g., cancers, such as melanoma; lung cancers; pancreatic cancer; stomach, colon and rectal cancers; prostate cancer; breast cancer; leukemias and lymphomas; and the like, said method comprising administering to a human or other mammal in need there of a therapeutically effective amount of the lipid-modified phosphodiester nucleoside prodrugs of the invention. The present invention provides anti-cancer lipid-modified phosphodiester nucleoside prodrugs as compounds of this invention which include, but are not limited to, cytarabine (ara-C), fluorouridine, fluorodeoxyuridine (floxuridine), gemcitibine, cladribine, fludarabine, pentostatin (2′-deoxycoformycin), 6-mercaptopurine and 6-thioguanine and substituted or unsubstituted ara-adenosine (ara-A), ara-guanosine (ara-G), and ara-uridine (ara-U). Anticancer compounds of the invention may be used alone or in combination with other antimetabobtes or with other classes of anticancer drugs such as alkaloids, topoisomerase inhibitors, alkylating agents, antifumor antibiotics, and the like.

In another aspect, the present invention provides a method of treatment of disease that uses a lipid-modified phosphodiester nucleoside prodrug in combination with another therapeutic drug, said method comprising administering to a human or other mammal in need there of a therapeutically effective amount of the lipid-modified phosphodiester nucleoside prodrugs of the invention in combination with another therapeutic drug. Illustrative examples of lipid-modified phosphodiester nucleoside prodrugs combination with other therapeutics, dosage unit forms, and amounts suitable for use in the methods and compositions of the present invention are listed in Table 3.

TABLE 3
Combinations and dosages for administration of lipid-modified phosphodiester
nucleoside prodrugs with other therapeutics.

| | | | Dose amount; | | Dosage |
|---|---|---|---|---|---|
| Combo | Indication | API's | Admin Method | | Schedule |
| | | Plus Interferons | | | |
| 1. | Hepatitis C | ODE-Phosphodiester Ribavirin Prodrug; | 1200 | mg po; | QD for 48 weeks; |
| | | PEGASYS (peginterferon alfa-2a) | 180 | μg sc | QW for 48 weeks |
| 2. | Hepatitis C | ODE-Phosphodiester Ribavirin Prodrug; | 800 | mg po; | QD for 48 weeks; |
| | | Peg-Intron (pegylated interferon alfa-2b) | 1.5 | μg/kg sc | QW for 48 weeks |
| 3. | Hepatitis C | ODE-Phosphodiester Ribavirin Prodrug; | 1200 | mg po; | QD for 48 weeks; |
| | | Intron A (Interferon Alfa-2b) | 3 | MIU sc | TIW for 48 weeks |
| 4. | Hepatitis C | ODE-Phosphodiester Ribavirin Prodrug; | 1200 | mg po; | QD for 48 weeks; |
| | | Roferon A (Interferon Alfa-2a) | 3 | MIU sc | TIW for 48 weeks |
| 5. | Hepatitis C | ODE-Phosphodiester Ribavirin Prodrug; | 1200 | mg po; | QD for 48 weeks; |
| | | Infergen (Consensus Interferon) | 15 | μg sc | TIW for 48 weeks |
| | | Plus HCV Protease Inhibitors | | | |
| 6. | Hepatitis C | ODE-Phosphodiester Ribavirin Prodrug; | 1200 | mg po; | QD for 48 weeks; |
| | | Telaprevir (HCV protease inhibitor) | 750 | mg po | TID for 48 weeks |
| 7. | Hepatitis C | ODE-Phosphodiester Ribavirin Prodrug; | 1200 | mg po; | QD for 48 weeks; |
| | | Boceprevir (HCV protease inhibitor) | 800 | mg po | TID for 48 weeks |
| | | Plus HCV Polymerase Inhibitors | | | |
| 8. | Hepatitis C | ODE-Phosphodiester Ribavirin Prodrug; | 1200 | mg po; | QD for 48 weeks; |
| | | Valopicitabine (HCV polymerase | 400 | mg po | QD for 48 weeks |
| | | inhibitor) | | | |
| 9. | Hepatitis C | ODE-Phosphodiester Ribavirin Prodrug; | 1200 | mg po; | QD for 48 weeks; |
| | | R-1626 (HCV polymerase inhibitor) | 3000 | mg po | BID for 48 weeks |
| | | Plus Interferon plus Protease | | | |
| | | Inhibitors | | | |
| 10. | Hepatitis C | ODE-Phosphodiester Ribavirin Prodrug; | 1200 | mg po; | QD for 48 weeks; |
| | | PEGASYS (peginterferon alfa-2a); | 180 | μg sc; | QW for 48 weeks; |
| | | Telaprevir (HCV protease inhibitor); | 750 | mg po | TID for 48 weeks |
| 11. | Hepatitis C | ODE-Phosphodiester Ribavirin Prodrug; | 1200 | mg po; | QD for 48 weeks; |
| | | PEGASYS (peginterferon alfa-2a); | 180 | μg sc; | QW for 48 weeks; |
| | | Boceprevir (HCV protease inhibitor) | 800 | mg po | TID for 48 weeks |
| | | Plus Interferon plus Polymerase | | | |
| | | Inhibitors | | | |
| 12. | Hepatitis C | ODE-Phosphodiester Ribavirin Prodrug; | 1200 | mg po; | QD for 48 weeks; |
| | | PEGASYS (peginterferon alfa-2a); | 180 | μg sc; | QW for 48 weeks; |
| | | Valopicitabine (HCV polymerase | 400 | mg po | QD for 48 weeks |
| | | inhibitor) | | | |
| 13. | Hepatitis C | ODE-Phosphodiester Ribavirin Prodrug; | 1200 | mg po; | QD for 48 weeks; |
| | | PEGASYS (peginterferon alfa-2a); | 180 | μg sc; | QW for 48 weeks; |
| | | R-1626 (HCV polymerase inhibitor) | 3000 | mg po | BID for 48 weeks |
| | | Plus Neuraminidase Inhibitors | | | |
| 14. | Influenza | ODE-Phosphodiester Ribavirin Prodrug; | 1200 | mg po; | QD for 1 week; |
| | | Zanamivir (neuraminidase inhibitor) | 10 | mg | BID for 5 days |
| | | | | inhalation | |
| 15. | Influenza | ODE-Phosphodiester Ribavirin Prodrug; | 1200 | mg po; | QD for 1 week; |
| | | Oseltamivir (neuraminidase inhibitor) | 75 | mg po | BID for 5 days |
| | | Plus M2 Ion Channel Blockers | | | |
| 16. | Influenza | ODE-Phosphodiester Ribavirin Prodrug; | 1200 | mg po; | QD for 1 week; |
| | | Amantadine (M2 ion channel blocker) | 200 | mg po | QD for 5 days |
| 17. | Influenza | ODE-Phosphodiester Ribavirin Prodrug; | 1200 | mg po; | QD for 1 week; |
| | | Rimantadine (M2 ion channel blocker) | 200 | mg po | QD for 5 days |

In some embodiments, this invention provides a method of treatment of hepatitis C (HCV) employing administering a octadecyl-ethanediol-modified (ODE) phosphodiester ribavirin prodrug to a patient in combination with PEGASYS (peginterferon alfa-2a). In other embodiments, octadecyl-ethanediol-modified (ODE) phosphodiester ribavirin prodrug is administered orally (po) in a dosage unit of about 100 mg to 4000 mg per day once a day (qd) concomitantly with peginterferon alfa-2a administered subcutaneously (SC) in a dosage unit of about 180 μg once a week for 48 weeks to adults with detectable hepatitis C virus and compensated liver disease. The actual dose administered varies depending on a number of patient factors including patient weight. The octadecyl-ethanediol-modified (ODE) phosphodiester ribavirin prodrug of this invention provides an improved therapeutic index relative to the parent drug ribavirin through a reduction in toxic side effect of hemolytic anemia coupled with improved efficacy through selective distribution to the liver, release of the active phosphorylated form of ribavirin, and reduced intracellular catabolism in treated tissue.

In some embodiments, this invention provides a method of treatment of hepatitis C (HCV) employing octadecyl-ethanediol-modified (ODE) phosphodiester ribavirin prodrug co-administered with Peg-Intron (pegylated interferon alfa-2b). In other embodiments, octadecyl-ethanediol-modified (ODE) phosphodiester ribavirin prodrug is administered orally (po) in a dosage unit of about 100 mg to 4000 mg per day once a day (qd) concomitantly with pegylated interferon alfa-2b administered subcutaneously (SC) in a dosage unit of about 15 μg/kg once a week for 48 weeks to adults with detectable hepatitis C virus and compensated liver disease. The actual dose administered varies depending on a number of patient factors including patient weight.

In some embodiments, this invention provides a method of treatment of hepatitis C (HCV) employing octadecyl-ethanediol-modified (ODE) phosphodiester ribavirin prodrug co-administered with interferon alfa-2b (Intron-A; REBETRON). In other embodiments, octadecyl-ethanediol-modified (ODE) phosphodiester ribavirin prodrug is administered orally (po) in a dosage unit of about 100 mg to 4000 mg per day once a day (qd) concomitantly with interferon alfa-2b administered subcutaneously (SC) in a dosage unit of about 3 million units (MIU) three times a week (TIW) for 48 weeks to adults with detectable hepatitis C virus and compensated liver disease. The actual dose administered varies depending on a number of patient factors including patient weight.

In some embodiments, this invention provides a method of treatment of hepatitis C (HCV) employing octadecyl-ethanediol-modified (ODE) phosphodiester ribavirin prodrug co-administered with interferon alfa-2a (Roferon). In other embodiments, octadecyl-ethanediol-modified (ODE) phosphodiester ribavirin prodrug is administered orally (po) in a dosage unit of about 100 mg to 4000 mg per day once a day (qd) concomitantly with interferon alfa-2a administered subcutaneously (SC) in a dosage unit of about 3 million units (MIU) three times a week (TIW) for 48 weeks to adults with detectable hepatitis C virus and compensated liver disease. The actual dose administered varies depending on a number of patient factors including patient weight.

In some embodiments, this invention provides a method of treatment of hepatitis C (HCV) employing octadecyl-ethanediol-modified (ODE) phosphodiester ribavirin prodrug co-administered with telaprevir (HCV protease inhibitor, VX-950). In other embodiments, octadecyl-ethanediol-modified (ODE) phosphodiester ribavirin prodrug is administered orally (po) in a dosage unit of about 100 mg to 4000 mg per day once a day (qd) concomitantly with telaprevir administered po in a dosage unit of about 100 mg to 4000 mg per day three times a day (tid) for 48 weeks to adults with detectable hepatitis C virus and compensated liver disease. The actual dose administered varies depending on a number of patient factors including patient weight.

In some embodiments, this invention provides a method of treatment of hepatitis C (HCV) employing octadecyl-ethanediol-modified (ODE) phosphodiester ribavirin prodrug co-administered with R-1626 (HCV polymerase inhibitor). In other embodiments, octadecyl-ethanediol-modified (ODE) phosphodiester ribavirin prodrug is administered orally (po) in a dosage unit of about 100 mg to 4000 mg per day once a day (qd) concomitantly with R-1626 administered po in a dosage unit of about 100 mg to 4000 mg per day twice a day (bid) for 48 weeks to adults with detectable hepatitis C virus and compensated liver disease. The actual dose administered varies depending on a number of patient factors including patient weight.

In some embodiments, this invention provides a method of treatment of hepatitis C (HCV) employing octadecyl-ethanediol-modified (ODE) phosphodiester ribavirin prodrug co-administered with PEGASYS (peginterferon alfa-2a) and telaprevir (HCV protease inhibitor). In other embodiments, octadecyl-ethanediol-modified (ODE) phosphodiester ribavirin prodrug is administered orally (po) in a dosage unit of about 100 mg to 4000 mg per day once a day (qd) concomitantly with peginterferon alfa-2a administered subcutaneously (SC) in a dosage unit of about 180 μg once a week and telaprevir administered po in a dosage unit of about 100 mg to 4000 mg per day three times a day (tid) for 48 weeks to adults with detectable hepatitis C virus and compensated liver disease. The actual dose administered varies depending on a number of patient factors including patient weight.

In some embodiments, this invention provides a method of treatment of hepatitis C (HCV) employing octadecyl-ethanediol-modified (ODE) phosphodiester ribavirin prodrug co-administered with PEGASYS (peginterferon alfa-2a) and R-1626 (HCV polymerase inhibitor). In other embodiments, octadecyl-ethanediol-modified (ODE) phosphodiester ribavirin prodrug is administered orally (po) in a dosage unit of about 100 mg to 4000 mg per day once a day (qd) concomitantly with peginterferon alfa-2a administered subcutaneously (SC) in a dosage unit of about 180 μg once a week and R-1626 administered po in a dosage unit of about 100 mg to 4000 mg per day twice a day (bid) for 48 weeks to adults with detectable hepatitis C virus and compensated liver disease. The actual dose administered varies depending on a number of patient factors including patient weight.

In another aspect, this invention provides a method of treatment of influenza employing octadecyl-ethanediol-modified (ODE) phosphodiester ribavirin prodrug co-administered with oseltamivir (TAMIFLU, neuraminidase inhibitor). In some embodiments, octadecyl-ethanediol-modified (ODE) phosphodiester ribavirin prodrug is administered orally (po) in a dosage unit of about 100 mg to 4000 mg per day once a day (qd) concomitantly with oseltamivir administered po in a dosage unit of about 10 mg to 2000 mg per day twice a day (bid) for about 7 days to adults for the treatment of uncomplicated acute illness due to influenza infection. The actual dose administered varies depending on a number of patient factors including patient weight.

In another aspect, this invention provides a method of treatment of influenza employing octadecyl-ethanediol-modified (ODE) phosphodiester ribavirin prodrug co-administered with rimantidine (M2 channel blocker). In some embodiments, octadecyl-ethanediol-modified (ODE) phosphodiester ribavirin prodrug is administered orally (po) in a dosage unit of about 100 mg to 4000 mg per day once a day (qd) concomitantly with oseltamivir administered po in a dosage unit of about 10 mg to 2000 mg per day once a day (qd) for about 7 days to adults for the treatment of uncomplicated acute illness due to influenza infection. The actual dose administered varies depending on a number of patient factors including patient weight.

The compositions and therapeutic combinations of the present invention are administered to a subject in need of antiviral treatment in a therapeutically effective amount to treat or prevent the viral infections. The daily dosage for the various compositions and therapeutic combinations described above can be administered to a subject in a single dose or in multiple subdoses, as desired. Subdoses can be administered 2 to 6 times per day, for example. Sustained release dosages can also be used, with less frequent administration. In some embodiments in which an lipid-modified phosphodiester nucleoside prodrugs and other antiviral therapeutics are administered in separate dosages, the number of doses of each component given per day may not necessarily be the same, e.g., one component may have a greater duration of activity and may therefore be administered less frequently.

The compositions and medicaments of the present invention can further comprise one or more pharmaceutically acceptable carriers, one or more excipients and/or one or more additives. The pharmaceutical compositions can comprise about 1 to about 99 weight percent of active ingredients, such as, for example, about 5 to about 95 percent active ingredients.

Useful pharmaceutically acceptable carriers can be solid, liquid or gas. Non-limiting examples of pharmaceutically acceptable carriers include solids and/or liquids such as magnesium carbonate, magnesium stearate, talc, sugar, lactose, ethanol, glycerol, water and the like. The amount of carrier in the unit dose form or formulation can range from about 5 to about 99 weight percent of the total weight of the treatment composition or therapeutic combination. Non-limiting examples of suitable pharmaceutically acceptable excipients and additives include non-toxic compatible fillers, binders such as starch, polyvinyl pyrrolidone or cellulose ethers, disintegrants such as sodium starch glycolate, crosslinked polyvinyl pyrrolidone or croscarmellose sodium, buffers, preservatives, anti-oxidants, lubricants, flavorings, thickeners, coloring agents, wetting agents such as sodium lauryl sulfate, emulsifiers and the like. The amount of excipient or additive can range from about 0.1 to about 95 weight percent of the total weight of the unit dose form or formulation. One skilled in the art would understand that the amount of carrier(s), excipients and additives (if present) can vary. Further examples of pharmaceutically acceptable carriers and methods of manufacture for various compositions can be found in A. Gennaro (ed.), Remington: The Science and Practice of Pharmacy, 21st Edition, (2005), Lippincott Williams & Wilkins, Baltimore, Md.

Useful solid form preparations for purposes of the present invention include powders, tablets, dispersible granules, capsules, cachets and suppositories. An example of a preparation of a preferred solid form dosage formulation is provided below.

Useful liquid form preparations for purposes of the present invention include solutions, suspensions and emulsions. Examples include water or water-propylene glycol solutions for parenteral injection. For oral solutions, suspensions and emulsions can contain sweetners and opacifiers. Liquid form preparations of the invention also include solutions for intranasal administration.

Aerosol preparations of the invention suitable for inhalation include solutions and solids in powder form, which may be in combination with a pharmaceutically acceptable carrier, such as an inert compressed gas, e.g., nitrogen.

The present invention includes solid form preparations which are intended to be converted, shortly before use, to liquid form preparations for either oral or parenteral administration. Such liquid forms include solutions, suspensions and emulsions.

The active pharmaceutical ingredients (“APIs” or “therapeutic agents”) employed in the methods and compositions of the invention can also be administered transdermally. The transdermal compositions can take the form of creams, lotions, aerosols and/or emulsions and can be included in a transdermal patch of the matrix or reservoir type, as are conventional in the art for other purposes.

In some embodiments, the APIs in the compositions and methods of this invention are administered orally. In other embodiments, the APIs in the compositions and methods of this invention are in a suitable oral dosage form. For example, the compositions of this invention can be compressed by usual methods into single or multi-layer tablets. Moreover, they can be produced in the form of coated tablets or provided in the form of hard-shell capsules. They can also be provided as oral suspensions or powders for reconstitution into oral suspensions. In general, the various oral dosage forms of the present compositions can be prepared by conventional procedures and techniques in view of the disclosure herein. The applicability of such methods and techniques to the formulation of the compositions of the present invention will be readily apparent to those skilled in the art in view of this disclosure.

In addition to the therapeutically active ingredients mentioned heretofore, the compositions of this invention can contain as optional ingredients any of the various diluents which are used ordinarily in the production of pharmaceutical preparations. Thus, for example, in formulating the present compositions into the desired oral dosage forms, one may use as optional ingredients any of the usual fillers, disintegrating agents or lubricating agents, e.g., lactose, gum arabic, starch, talc, magnesium or calcium stearate, gelatin, and the like. It should be fully understood, however, that the optional ingredients herein named are given by way of example only and that the invention is not restricted to the use thereof. On the contrary, other adjuvants such as preservatives, stabilizers, suspending agents or buffers, the identity and use of which are well known in the art, can and will be employed in carrying out this invention.

In practicing the methods above, co-administration in separate tablets or capsules, of representative formulations comprising a lipid-modified phosphodiester nucleoside prodrugs and another antiviral such as telaprevir, boceprevir, valopcitiabine, R-1626, osetamivir, amantidien, or rimantidine can be used.

The present invention also provides kits for antiviral treatment with a combination of active ingredients, wherein the active ingredients may be administered separately or as an admixture, and the invention also provides pharmaceutical compositions packaged in a kit optionally with instructions for use. The kit contains a pharmaceutical composition comprising at least one antiviral agent and a separate pharmaceutical composition comprising another antiviral or combination of antivirals or a single composition of an admixture of both, as described above, as well as, optionally, directions for the administration of the composition(s) contained in the kit. A kit can be advantageous, for example, when the separate components must be administered in different dosage forms (e.g., oral and parenteral) or are administered at different dosage intervals.

### IV. EXAMPLES

The invention will now be described in greater detail by reference to the following non-limiting examples. The following examples describe the preparation of ribavirin 5′-phosphodiester lipid prodrugs. A summary of the illustrative method provided by the invention for preparing compounds of the invention in synthetic preparation is shown in FIG. 1. With reference to FIG. 1, examples 1-7 describe the various steps of the synthesis. Those skilled in the art will recognize that the methods used in the examples described herein are readily applicable to the preparation of other related nucleoside phosphodiester lipid prodrugs as discussed in Section II.

### Example 1

### Synthesis of 1-O-octadecyl-ethanediol-2-dichlorophosphate (2)

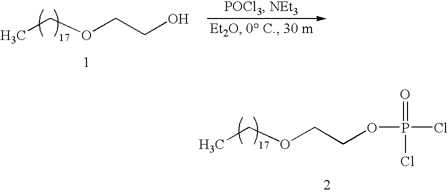

2-(Octadecyloxy)ethanol (1, 1.0 g, 3.18 mmol, 1 eq) is dissolved in dry ether (20 mL) and is cooled to 0° C. under N₂. Triethylamine (0.45 ml, 3.18 mmol, 1 eq) and POCl₃ (0.29 ml, 3.18 mmol, 1 eq) are added slowly. After stirring for 30 minutes at 0° C. under N₂, the reaction is filtered to remove the triethylamine hydrochloride salt, producing crude 2.

### Example 2

### Synthesis of 1-O-octadecyl-ethanediol-2-chlorophospho-ribavirin-2′,3′-acetonide (4)

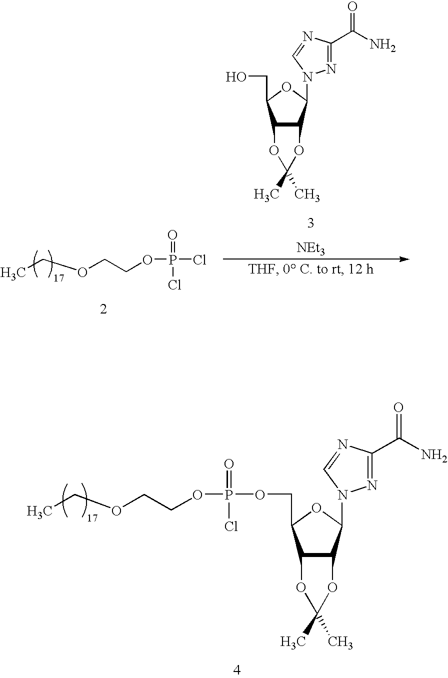

Dichlorophosphate 2 (1.0 g, 2.32 mmol, 1 eq) is dissolved in dry THF (15 mL), and the solution is cooled to 0° C. under N₂. Triethylamine (0.32 ml, 2.32 mmol, 1 eq) and ribavirin-2′,3′-acetonide (3, 0.66 g, 2.32 mmol, 1 eq) are added slowly. After stirring for 30 minutes at 0° C. under N₂, the reaction is allowed to warm to room temperature over 12 h. The reaction is filtered to remove the triethylamine hydrochloride salt, yielding crude 4.

### Example 3

### Synthesis of 1-O-octadecyl-ethanediol-2-phospho-ribavirin-2′,3′-acetonide (5)

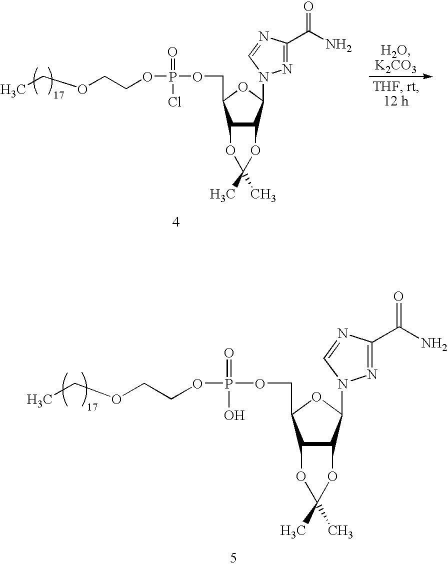

Chlorophosphate 4 (1.0 g, 1.47 mmol, 1 eq) is dissolved in THF (15 mL). Saturated aqueous K₂CO₃ (0.1 mL) is added and the reaction is stirred for 1 hour at room temperature. The reaction is concentrated in vacuo. The crude material is purified by flash chromatography (silica, gradient 70:30:3:3/CHCl₃:MeOH:NH₄OH:H₂O) to provide 5.

### Example 4

### Synthesis of 1-O-octadecyl-ethanediol-2-phospho-ribavirin (5)

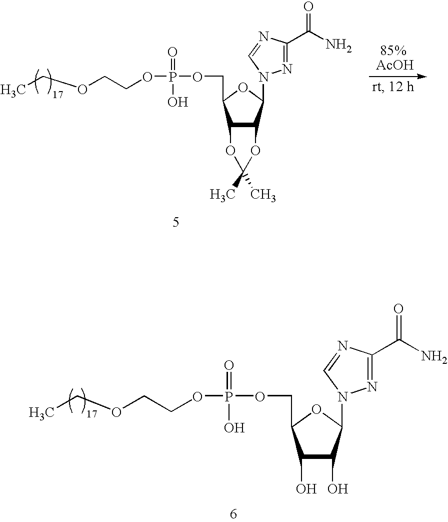

Acetonide 5 (1.0 g, 1.51 mmol, 1 eq) is treated with 85% AcOH (5 mL) and stirred for 12 h at room temperature. The reaction is concentrated in vacuo. The crude material is purified by flash chromatography (silica, gradient 70:30:3:3/CHCl₃:MeOH:NH₄OH:H₂O) to provide 6.

### Example 5

### Synthesis of 1-O-octadecyl-ethanediol-2-(2-cyanoethyl-N,N-diisopropyl)-phosphoramidite (8)

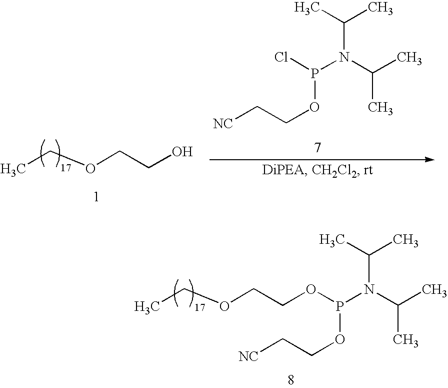

2-(Octadecyloxy)ethanol (1, 1.0 g, 3.18 mmol, 1 eq) is dissolved in dry CH₂Cl₂ (20 ml). Diisopropylethylamine (1.66 ml, 9.54 mmol, 3 eq) and 2-cyanoethyl-N,N-diisopropylphosphoramidochlorite (0.99 ml, 4.45 mmol, 1.4 eq) are added dropwise under N₂. After stirring for 1 hour at room temperature under N₂, the reaction is diluted with ethyl acetate (250 ml), washed with brine, dried over Na₂SO₄, and concentrated in vacuo. The crude material is purified by flash chromatography (silica, 1:1/hexane:Et₂O+1% NEt₃) to provide 8.

### Example 6

### Synthesis of 1-O-octadecyl-ethanediol-2-(2-cyanoethyl)-5′-ribavirin-phosphotriester (9)

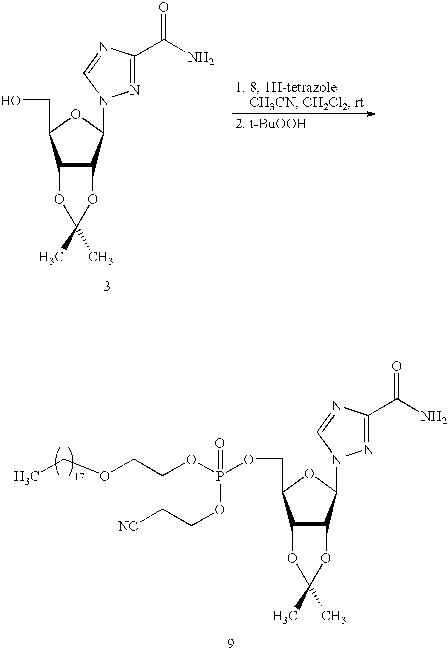

Ribavirin-2′,3′-acetonide (3, 1.0 g, 3.52 mmol, 1 eq) is dissolved in dry CH₃CN:CH₂Cl₂/1:1 (20 ml). Phosphoramidite 8 (1.81 g, 3.52 mmol, 1 eq) and 1-H-tetrazole (0.74 g, 10.56 mmol, 3 eq) are added under N₂, and the reaction is stirring for 24 hour at room temperature under N₂.

t-BuOOH (5.5 M in decane, 2.56 ml, 14.08 mmol, 4 eq) is added and the reaction is stirred at room temperature for 1 h. The reaction is partitioned between CHCl₃ and saturated Na₂S₂O₃, is extracted with CHCl₃, is washed with brine, is dried over Na₂SO₄, and concentrated in vacuo. The crude material is purified by flash chromatography (silica, 25% EtOAc:Hexane) to provide 9.

### Example 7

### Synthesis of 1-O-octadecyl-ethanediol-2-phospho-ribavirin-2′,3′-acetonide (5)

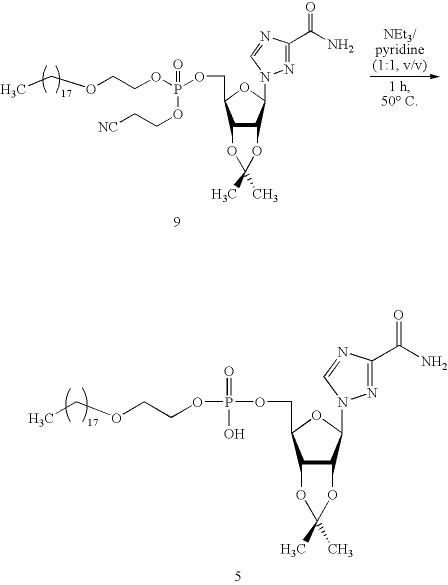

Triester 9 (1.0 g, 1.4 mmol, 1 eq) is treated with NEt₃/pyridine (1:1, 10 mL) and stirred at room temperature for 12 h. The reaction is concentrated in vacuo. The crude material is purified by flash chromatography (silica, gradient 70:30:3:3/CHCl₃:MeOH:NH₄OH:H₂O) to provide 5.

### Example 8

### 1-(2,3-O-isopropylidene-beta-D-ribofuranosyl)-1H-1,2,4-triazole-3-carboxamide (10)

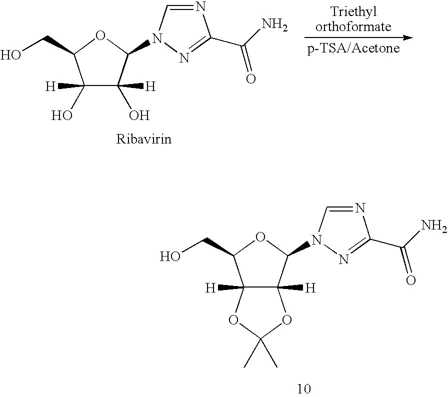

Triethyl orthoformate (5.99 mL/36.0 mmol) and p-toluenesulfonic acid (0.068 g/0.360 mmol) were added to acetone (40 mL) and the reaction was allowed to stir at room temperature overnight. The resulting red solution was added to a suspension of Ribavirin (4.00 g/16.4 mmol) in dry DMF (10 mL). The red color mostly vanished. The suspension was stirred for 12 h at 50° C. and then overnight at room temperature. The reaction was concentrated in vacuo to give a viscous yellow residue. The residue was re-dissolved in THF. Silica gel was added to the THF solution and the suspension was concentrated in vacuo. The residue was placed on top of a 90 g silica gel cartridge and the column was eluted sequentially with dichloromethane (400 mL), then 5% methanol in dichloromethane (1 L) and finally 10% methanol in dichloromethane (1 L). Like fractions of pure product were combined and concentrated in vacuo. The residue was suspended in chloroform and concentrated in vacuo to give the title compound 10 (4.02 g/86%) as a white solid. ¹H NMR (400 MHz, DMSO-d₆) δ ppm 1.33 (s, 3H) 1.51 (s, 3H) 3.36-3.53 (m, 2H) 4.23 (dt, J=6.06, 1.76 Hz, 1H) 4.91 (dd, J=6.01, 1.87 Hz, 1H) 4.94-5.01 (m, 1H) 5.19 (dd, J=6.01, 1.45 Hz, 1H) 6.21 (d, J=1.45 Hz, 1H) 7.66 (s, 1H) 7.86 (s, 1H) 8.81 (s, 1H). MS ES⁺ m/z 307.2 (M+Na)⁺, 285.3 (M+H)⁺. MS ES⁻ m/z 283.3 (M−H)⁺.

### Example 9

### 1-{5-O-[(2-chlorophenoxy)(octadecyloxy)phosphoryl]-2,3-O-isopropylidene-beta-D-ribofuranosyl}-1′-1H-1,2,4-triazole-3-carboxamide (11)

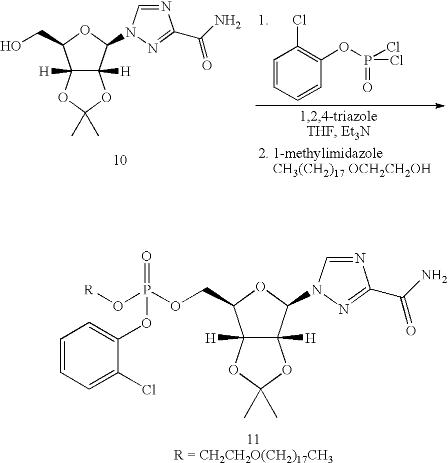

Into a solution of triazole (0.146 g/2.12 mmol), triethylamine (0.215 g/2.12 mmol) and dry THF (2.1 mL) was added a solution of 2-chlorophenyl phosphorodichloridate (0.259 g/1.06 mmol) dissolved in dry THF (1.3 mL). A white solid formed. The reaction was stirred at room temperature for 1 h and then filtered. The filter pad was washed with dry THF (2.1 mL). To the filtrate was added additional THF (1.2 mL), 1-(2,3-O-isopropylidene-beta-D-ribofuranosyl)-1H-1,2,4-triazole-3-carboxamide (0.226 g/0.795 mmol) 10 and 1-methylimidazole (0.084 mL/1.06 mmol). The reaction was stirred at room temperature for 1 h, then 2-(octadecyloxy)ethanol (0.250 g/0.795 mmol) was added, and the reaction was stirred at room temperature overnight. The reaction was concentrated in vacuo and the residue was dissolved in dichloromethane and loaded onto a 40 g silica gel cartridge that had been pre-equilibrated with dichloromethane. The column was eluted sequentially with dichloromethane (100 ml), then 2.5% methanol in dichloromethane (250 mL) and finally 5% methanol in dichloromethane. A poor separation was obtained and all fractions containing product were combined and concentrated in vacuo. The residue was re-dissolved in dichloromethane and loaded on top of a 40 g silica gel cartridge that had been pre-equilibrated with dichloromethane. The column was eluted sequentially with dichloromethane (250 mL), then 1% methanol in dichloromethane (250 mL), followed by 2% methanol in dichloromethane (250 mL) and finally 4% methanol in dichloromethane. Like fractions of pure product were combined and concentrated in vacuo. Residue was dissolved in dichloromethane and concentrated in vacuo to give the title compound 11 (0.439 g/71%) as a colorless viscous oil. ¹H NMR (400 MHz, DMSO-d₆) δ ppm 0.80-0.90 (m, 3H) 1.15-1.30 (m, 30H) 1.33 (s, 3H) 1.39-1.48 (m, 2H) 1.51 (s, 3H) 3.29-3.39 (m, 2H) 3.50-3.59 (m, 2H) 4.13-4.30 (m, 3H) 4.31-4.41 (m, 1H) 4.43-4.51 (m, 1H) 5.02 (dd, J=5.81, 2.28 Hz, 1H) 5.12-5.18 (m, 1H) 6.38 (d, J=5.60 Hz, 1H) 7.20-7.27 (m, 1H) 7.27-7.39 (m, 2H) 7.51-7.58 (m, 1H) 7.67 (s, 1H) 7.86 (br. s., 1H) 8.81 (s, 1H). MS ES⁺ m/z 793.9 (M+Na)⁺.

### Example 10

### 1-(5-O-{hydroxy[2-(octadecyloxy)ethoxy]phosphoryl}-2,3-O-isopropylidene-beta-D-ribofuranosyl)-1H-1,2,4-triazole-3-carboxamide (12)

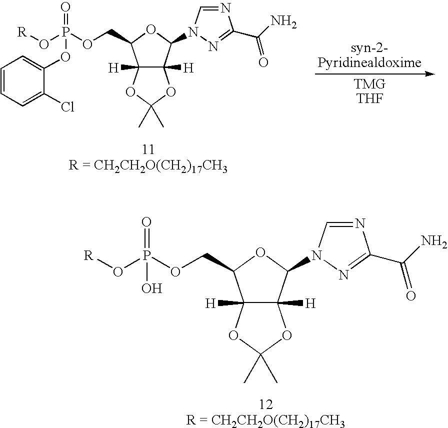

Into a solution of 1-{5-O-[(2-chlorophenoxy)(octadecyloxy)phosphoryl]-2,3-O-isopropylidene-beta-D-ribofuranosyl}-1H-1,2,4-triazole-3-carboxamide 11 (0.448 g/0.581 mmol) and dry THF (8.0 mL) was added a solution of 1,1,3,3-tetramethylguanidine (0.378 g/3.28 mmol) and syn-2-pyridinealdoxime (0.401 g/3.28 mmol) dissolved in dry THF (4.2 mL). The reaction was diluted with additional dry THF (4.2 mL) and stirred at room temperature overnight. The reaction was then concentrated in vacuo and the residue was dissolved in dichloromethane and loaded onto a 40 g silica gel cartridge that had been pre-equilibrated with dichloromethane. The column was eluted sequentially with dichloromethane (40 mL), 10% methanol in dichloromethane (250 mL), 20% methanol in dichloromethane (250 mL) and finally 30% methanol in dichloromethane. Like fractions of pure product were combined and concentrated in vacuo to give (0.368 g/96%) of the title compound which contained trace amounts of 1,1,3,3,-tetramethylgaunidine. This contaminated material (0.345 g) was dissolved in a 1:1 solution of THF and ethyl acetate (15 mL) and water (5 mL) was added to the solution. The resulting biphasic mixture was cooled in an ice/water bath and the aqueous phase was acidified to pH=1-2 with cold 1M HCl. The acidified mixture was shaken and then kept cold while the layers separated. The organic layer was isolated and the aqueous layer was diluted with water (5 mL). The aqueous layer was washed two additional times with a 1:1 solution of THF and ethyl acetate. The organic layers were combined, dried with sodium sulfate and concentrated in vacuo. The residue was dissolved in methanol and concentrated in vacuo to help remove residual water. This process was repeated three more times. The residue was dissolved in dichloromethane and concentrated in vacuo to give (0.312 g/81%) of the title compound 12 as a white solid. ¹H NMR (400 MHz, DMSO-d₆) δ ppm 0.85 (t, J=6.70 Hz, 3H) 1.17-1.29 (m, 30H) 1.33 (s, 3H) 1.46 (t, J=6.43 Hz, 2H) 1.51 (s, 3H) 3.35 (t, J=6.63 Hz, 2H) 3.47 (t, J=4.46 Hz, 2H) 3.84-3.98 (m, 3H) 3.98-4.08 (m, 1H) 4.40 (dt, J=6.43, 2.07 Hz, 1H) 5.00 (dd, J=5.91, 2.18 Hz, 1H) 5.13-5.19 (m, 1H) 6.30-6.37 (m, 1H) 7.67 (s, 1H) 7.91 (s, 1H) 8.81 (s, 1H). MS ES⁺ m/z 661.5 (M+H)⁺. MS ES⁻ m/z 659.6 (M−H)⁺. HPLC=100%.

### Example 11

### 1-(5-O-{hydroxy[2-(octadecyloxy)ethoxy]phosphoryl}-beta-D-ribofuranosyl)-1H-1,2,4-triazole-3-carboxamide (13)

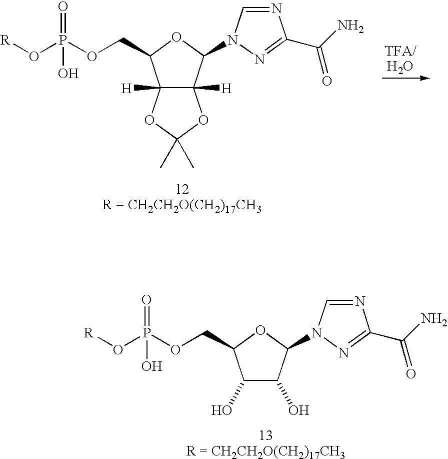

The acetonide, 1-(5-O-{hydroxy[2-(octadecyloxy)ethoxy]phosphoryl}-2,3-O-isopropylidene-beta-D-ribofuranosyl)-1H-1,2,4-triazole-3-carboxamide, 12 (0.304 g/0.460 mmol) was dissolved in a 9:1 mixture of trifluoroacetic acid and water (4 mL) and stirred at room temperature. After stirring for 45 min the reaction was concentrated in vacuo. Toluene was added to the residue and the mixture was concentrated in vacuo. This process was repeated several times to azeotrope residual water from the residue. Methanol was added to the residue and the suspension concentrated. This process was repeated several times until a concentration in vacuo yielded a white solid. The white solid was dissolved in dichloromethane and concentrated in vacuo to give (0.264 g/93% of the title compound as a white solid: ¹H NMR (400 MHz, DMSO-d₆) □ ppm 0.81-0.91 (m, 3H) 1.16-1.33 (m, 30H) 1.46 (t, J=6.22 Hz, 2H) 3.34 (t, J=6.63 Hz, 2H) 3.47 (t, J=4.46 Hz, 2H) 3.86-4.03 (m, 3H) 4.05-4.16 (m, 2H) 4.19-4.26 (m, 1H) 4.31-4.38 (m, 1H) 5.88 (d, J=3.52 Hz, 1H) 7.64 (s, 1H) 7.87 (s, 1H) 8.82 (s, 1H). MS ES⁺ m/z 659.4 (M+K)⁺, 643.4 (M+Na)⁺, 621.4 (M+H)⁺. MS ES⁻ m/z 619.5 (M−H)⁺. HPLC=100%.

### Example 12

### Assay for In Vitro Red Blood ATP Levels

Red blood ATP levels are used as a surrogate marker to demonstrate a compound's potential to cause hemolytic anemia, an undesirable side effect. To determine the effect of octadecyl-ethanediol-modified (ODE) phosphodiester ribavirin prodrug on red blood cell ATP levels, in particular the, washed red cells are incubated at 10% hematocrit in a buffer containing 120 mmol/L NaCl, 5 mmol/L KCl, 1.2 mmol/L MgSO₄, 1.2 mmol/L KH₂PO₄, 24 mmol/L NaHCO₃, pH 7.4, supplemented with 50% plasma, and 10 mmol/L glucose, with and without the ribavirin phospholipid prodrug or ribavirin (1 mmol/L). After 12 hours incubation at 37° C., the red cells are washed 4 times in phosphate-buffered saline (PBS) and immediately used for different measurements. Analysis of red cell ATP level in neutralized perchloric acid extracts is performed by standard spectrophotometric methods. Differences in ATP levels are correlated to hemolytic effects.

### Example 13

### Assay for In Vitro Efficacy Against HCV Replicon

The HCV RNA replicon assay utilizes the cell line Huh7 ET (luc-ubi-neo/ET), which contains a HCV RNA replicon with a stable luciferase (LUC) reporter (Murray, M; Korba, B “Hepatitis C Virus Assays”, http://niaid-aacf.org/protocols/HCV.htm). The LUC reporter is used as an indirect measure of HCV replication. The activity of the LUC reporter is directly proportional to HCV RNA levels and positive control antiviral compounds behave comparably using either LUC or RNA endpoints. The HCV RNA replicon assay is used to examine the effects of the octadecyl-ethanediol-modified (ODE) phosphodiester ribavirin prodrug at five half-log concentrations each. Human interferon alpha-2b is included in each run as a positive control compound. Subconfluent cultures of the ET line are plated out into 96-well plates that are dedicated for the analysis of cell numbers (cytotoxicity) or antiviral activity and the next day drugs are added to the appropriate wells. Cells are processed 72 hr later when the cells are still subconfluent. ODE-phosphodiester ribavirin prodrug EC₅₀ and EC₉₀ values (antiviral activity) are derived from HCV RNA levels assessed as either HCV RNA replicon-derived LUC activity or as HCV RNA using TaqMan RT-PCR. ODE-phosphodiester ribavirin prodrug IC₅₀ and IC₉₀ values (cytotoxicity) are calculated using CytoTox-1 (Promega), a colorimetric assay used as an indicator of cell numbers and cytotoxicity when the LUC assay system is employed, while ribosomal (rRNA) levels determined via TaqMan RT-PCR are used as an indication of cell numbers in the RNA-based assay.

### Example 14

### Evaluation of Octadecyl-Ethanediol-Modified (ODE) Phosphodiester Ribavirin Prodrug for the Inhibition of HCV Replication in Cell Culture

Antiviral activity of the test compounds was assessed (Okuse et al., 2005, Antivir. Res. 65:23) in the stably HCV replicating line, AVA5 (genotype 1b, subgenomic, replicon, Blight et al., 2000, Sci. 290: 1972) and APC 103 ((genotype 1a, genomic replicon). (ODE) phosphodiester ribavirin prodrug were added to dividing cultures daily for three days. Cultures generally start the assay at 30-50% confluence and reach confluence during the last day of treatment. Intracellular HCV RNA levels and cytotoxicity (on 96 well plates) were used. A total of 12 untreated control cultures and triplicate cultures treated with α-interferon and 2′CmeC served as assay controls.

Triplicate cultures for HCV RNA levels were measured using a conventional blot hybridization method in which HCV RNA levels were normalized to the levels of β-actin RNA in each individual culture (Okuse et al., 2005, Antivir. Res. 65:23). Cytotoxicity was measured using an established neutral red dye uptake assay (Korba et al., 1992, Antivir. Res. 19:55, Okuse et al., Antivir. Res. 65:23). Test compounds were received as powders on dry ice and were dissolved in 100% tissue culture grade DMSO (Sigma, Inc.) at 10 mM. Aliquots of test compounds sufficient for one daily treatment were made in individual tubes and all material was stored at −20° C. On each day of treatment daily aliquots were suspended into culture medium at room temperature and immediately added to cell cultures.

(ODE) phosphodiester ribavirin prodrug induced selective reductions in intracellular HCV RNA levels produced by AVA5 and APC103 cultures at the concentrations tested. Significant toxicity for (greater than 50% depression of the dye uptake levels observed in untreated cells) was observed for ribavirin at the concentrations used for the antiviral analyses. No significant toxicity was observed for (ODE) phosphodiester ribavirin prodrug.

### Example 15

### Pharmacokinetic Study of (ODE) Phosphodiester Ribavirin Prodrug Following Single Oral and Intravenous Administration in Male CD-1 Mice

The purpose of this study was to evaluate the pharmacokinetic (PK) profile of (ODE) phosphodiester ribavirin prodrug after single oral (P.O.) and intravenous (I.V.) administration in male CD-1 mice.

Forty-eight male CD-1 mice selected for the study were divided into three study groups, 6 mice in Group 1 without treatment and for pre-dose PK sample collection, 21 mice in Group 2 for the oral administration of (ODE) phosphodiester ribavirin prodrug at 30 mg/kg, and 21 mice in Group 3 for the intravenous administration of (ODE) phosphodiester ribavirin prodrug at 5 mg/kg. Plasma samples were collected at pre-dose, 1, 3, 6, 12, 24, 48, and 72 hours post-dose for P.O. treatment group and at pre-dose, 0.25, 2, 6, 12, 24, 48, and 72 hours post-dose for I.V. treatment group. All samples were collected within ±2 minutes of the targeted time.

The LC-MS/MS method for the quantitative determination of (ODE) phosphodiester ribavirin prodrug in the mouse plasma was developed. Phenolphthalein was used as an internal standard. The method is specific for (ODE) phosphodiester ribavirin prodrug in the mouse plasma and the concentration-response relationship was linear in the calibration range of 5.0 to 500 ng/mL with a regression coefficient equal to or greater than 0.9981. The sample processing included the addition of 50 μL of 1:1 acetonitrile:water (or (ODE) phosphodiester ribavirin prodrug working solutions for calibration standards and QC samples), 50 μL of 500 ng/mL internal standard and 150 μL acetonitrile into 50 μL mouse plasma (or blank pooled mouse plasma for calibration standards and QC samples). The samples were mixed by vortexing for 5 minutes, and centrifuged at 15,000 rpm at 4° C. for 10 minutes. A 150-μL aliquot of the supernatant was transferred to a 96-well plate and 10 uL of supernatant sample was injected into the LC-MS/MS system for analysis. In each analytical batch, PK samples were run concurrently with calibration standards (blank, 0, 5, 10, 25, 50, 100, 150, 300, and 500 ng/mL), and low, mid, high, and 10-fold dilution QC samples (15, 250, 400, and 2000 ng/m L).

Pharmacokinetic parameters of (ODE) phosphodiester ribavirin prodrug in mouse plasma were obtained using non-compartmental model (WinNonlin Professional, version 5.0.1). A 1/Y² weighting factor was used.

Following intravenous administration of (ODE) phosphodiester ribavirin prodrug at 5 mg/kg, the Tₘₐₓ and Cₘₐₓ were observed at 0.25 hours and 1960 ng/mL, respectively. (ODE) phosphodiester ribavirin prodrug had a plasma half-life time of 1.13 hours. The value of AUC_{0→∞} was calculated to be 2659 hr·ng/mL. The total clearance (Cl) and the volume of distribution (Vss) at the steady-state were obtained to be 1881 mL/hr/kg and 913 mL/kg, respectively.

Following the oral administration of (ODE) phosphodiester ribavirin prodrug at 30 mg/kg, the Tₘₐₓ and Cₘₐₓ were observed at 3.0 hours and 407 ng/mL, respectively. The value of AUC_{0→∞} was calculated to be 1705 hr·ng/mL. The oral bioavailability of (ODE) phosphodiester ribavirin prodrug was calculated to be 10.7%.

(ODE) phosphodiester ribavirin prodrug plasma levels were below LLOQ (5 ng/mL) from 24 hours to 72 hours following the oral administration, and from 12 hours to 72 hours following the intravenous administration.

During in-life phase, cageside observations were performed twice daily and detailed clinical observations were performed prior to dosing.

TABLE 4
Non-compartmental pharmacokinetic parameters of
(ODE) phosphodiester ribavirin prodrug in mouse
plasma following the intravenous and oral administration

| Parameter | IV | PO |
|---|---|---|
| Dose (mg/kg) | 5 | 30 |
| Tₘₐₓ (hr) | 0.25 | 3.0 |
| Cₘₐₓ (ng/mL) | 1960 | 407 |
| t_{1/2} (hr) | 1.13 | Not available |
| CI (mL/hr/kg) | 1881 | Not applicable |
| Vss (mL/kg) | 913 | Not applicable |
| AUC_{0→tlast} (hr · ng/mL) | 2645 | 1661 |
| AUC_{0→∞} (hr · ng/mL) | 2659 | 1705 |
| F (%) | — | 10.7 |

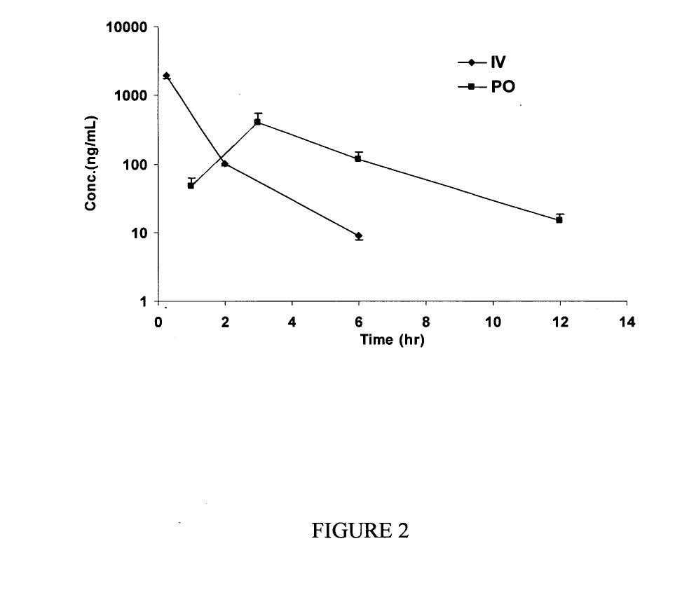

FIG. 2 shows the pharmacokinetics of (ODE) phosphodiester ribavirin prodrug in mouse plasma following intravenous administration of 5 mg/kg and oral administration of 30 mg/kg in male mice (N=3).

### Example 15

### Evaluation of Octadecyl-ethanediol-modified (ODE) Phosphodiester Ribavirin Prodrug for Cytotoxicity against HepG2, CEM and PBMC cells

CytoTox-ONE™ homogeneous membrane integrity assay kits (Promega) was used in the cytotoxicity studies. The assay measured the release of lactate dehyrodegenase (LDH) from cells with damaged membranes in a fluorometric, homogeneous format. LDH released into the culture medium was measured with a coupled enzymatic assay that resulted in the conversion of resazurin into a fluorescence resorufin product. The amount of fluorescence produced is proportional to the number of lysed cells. The cytotoxicity assessment assay was performed using a designed plate format using a designed plate format for allocation of media, drug, cells, and virus in a 96 well plate. Six serially diluted concentrations of octadecyl-ethanediol-modified (ODE) phosphodiester Ribavirin prodrug was applied to the cells to derive the TC₅₀ (toxic concentration of the drug decreasing the cell viability by 50%), TC₉₀ (toxic concentration of the drug decreasing the cell viability by 90%) and TC₉₅ (toxic concentration of the drug decreasing the cell viability by 95%) values. The octadecyl-ethanediol-modified (ODE) phosphodiester Ribavirin prodrug had TC₅₀, TC₉₀ and TC₉₅ values of 13.15 μM, 28.32 μM and 41.69 μM, respectively. against HepG2 cells. The octadecyl-ethanediol-modified (ODE) phosphodiester Ribavirin prodrug had TC₅₀, TC₉₀ and TC₉₅ values of 14.06 μM, 63.41 μM and 84.55 μM, respectively. against CEM cells. The octadecyl-ethanediol-modified (ODE) phosphodiester Ribavirin prodrug had TC₅₀, TC₉₀ and TC₉₅ values of 111 μM, 245 μM and 272 μM, respectively. against PBMC cells.

### Example 16

### Evaluation of Octadecyl-ethanediol-modified (ODE) Phosphodiester Ribavirin Prodrug Activity Against Influenza A

A CPE (virus induced cytopathogenic effects) inhibition assay procedure was employed to evaluate octadecyl-ethanediol-modified (ODE) phosphodiester Ribavirin prodrug for antiviral activity against influenza A in Madin-Darby canine kidney (MDCK) cells. The antiviral assay was designed to test six concentrations of octadecyl-ethanediol-modified (ODE) phosphodiester Ribavirin prodrug in triplicate against influenza A. Cell controls containing medium alone. virus infected cell controls containing medium and virus, drug cytotoxicity controls containing medium and each drug concentration, reagent controls containing culture medium only (no cells) and drug calorimetric controls containing drug and medium (no cells) were run simultaneously with the test samples. Ribavirin was used as positive control compound. The plates were incubated at 37° C. in a humidified atmosphere containing 5% CO₂ until maximum CPE is observed in the untreated virus control cultures. Inhibition of CPE by ODE) phosphodiester Ribavirin prodrug was determined using Cell Titer®AQuₑₒᵤₛ One Solution Cell Proliferation assay (Promega) which is a colorimetric method for determining the number of viable cells. The reagent contains a novel tetrazolium compound, MTS, and an electron coupling agent, PES, which when combined form a stable solution. The MTS tetrazolium compound is bioreduced by NADPH or NADH produced by dehydrogenase in metabolically active cells. Therefore the quantity of formazan product measures is directly proportional to the number of living cells in culture. A computer program is utilized to calculate the percent of CPE reduction of the virus infected cells and the percentage viability of uninfected drug control wells. The minimum inhibitory drug concentration which reduces the CPE by 50% (IC₅₀) and the minimum toxic drug concentration which causes the reduction of viable cells by 50% (TC₅₀) were calculated. A therapeutic index (TI₅₀) was determined by dividing the TC₅₀ by the IC₅₀. The measured IC₅₀ for octadecyl-ethanediol-modified (ODE) phosphodiester Ribavirin prodrug was less than 0.316 μM while the TC₅₀ was 66.4 μM and the TI was greater than 210.

Although the foregoing invention has been described in some detail by way of illustration and example for purposes of clarity and understanding, it will be apparent to those of ordinary skill in the art in light of the teaching of this invention that certain changes and modifications may be made thereto without departing from the spirit or scope of the claims.

1. A lipid-modified phosphodiester nucleoside prodrug compound comprising a phosphorylated nucleoside analog covalently linked to a lipid.

2. The compound of claim 1 having the structure of formula: 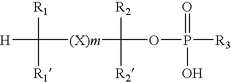
wherein R₁ and R₁′ are independently hydrogen, substituted and unsubstituted —O(C₁-C₂₄)alkyl, —O(C₁-C₂₄)alkenyl, —O(C₁-C₂₄)acyl, —S(C₁-C₂₄)alkyl, —S(C₁-C₂₄)alkenyl, or —S(C₁-C₂₄)acyl, wherein at least one of R₁ and R₁′ is not hydrogen, and wherein said alkenyl or acyl moieties have 1 to 6 double bonds;
wherein R₂ and R₂′ are independently hydrogen, substituted and unsubstituted —O(C₁-C₇)alkyl, —O(C₁-C₇)alkenyl, —S(C₁-C₇)alkyl, —S(C₁-C₇)alkenyl, —O(C₁-C₇)acyl, —S(C₁-C₇)acyl, —N(C₁-C₇)acyl, —NH(C₁-C₇)alkyl, —N((C₁-C₇)alkyl)₂, oxo, halogen, —NH₂, —OH, or —SH;
wherein R₃ is a nucleoside comprising a ribose or a modified ring or non-ring structure linked to the phosphorus via a phosphoester bond; and
wherein X is carbon and m is an integer from 0 to 6.

3. The compound of claim 2 wherein m=0, 1 or 2 and R₂ and R₂′ comprise H.

4. The compound of claim 3 having the structure:

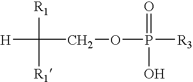

5. The compound of claim 3 having the structure:

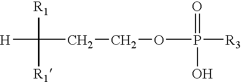

6. The compound of claim 3 wherein the glycerol phosphate species has the structure:

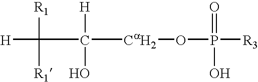

7. The compound of claim 2 wherein R₁ is −0(C₁-C₂₄)alkyl.

8. The compound of claim 2 wherein R₁ᵢₛ—O(C₁₂-C₁₉)alkyl.

9. The compound of claim 2 wherein R₁ is —O(C₁₆-C₁₇)alkyl.

10. A method of treatment of a viral infection said method comprising administering to a subject in need of treatment a therapeutically effective amount of a lipid-modified phosphodiester nucleoside prodrug of claim 1.

11. The method of claim 10 wherein the amount administered is between 0.01 and 1,000 mg/kg/day.

12. The method of claim 10 wherein the amount administered is between 0.10 and 100 mg/kg/day.

13. The method of claim 10 wherein the viral infection is a hepatitis C infection and said prodrug has the structure:

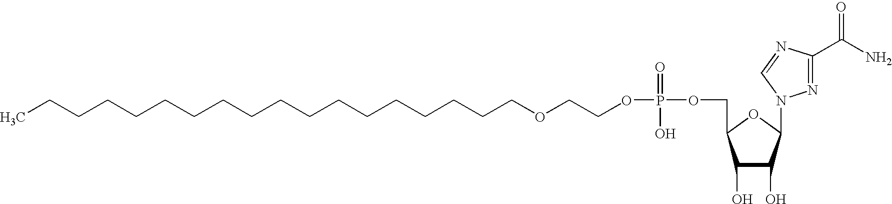

14. The method of claim 13 wherein the therapeutically effective amount is a dose between 100 and 4000 mg/day.

15. The method of claim 10 wherein the viral infection is a respiratory syncytial virus infection and said prodrug has the structure:

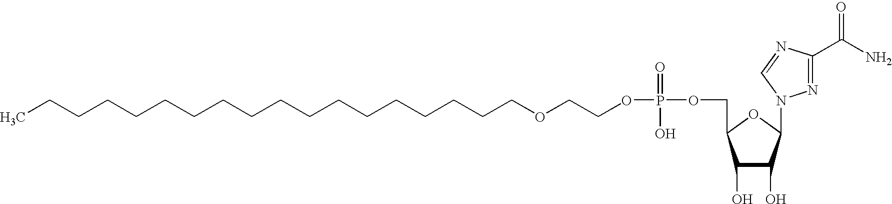

16. The method of claim 15 wherein the therapeutically effective amount is a dose between 100 and 4000 mg every 6 hours for 4 days, followed by a dose between 100 and 4000 mg every 8 hours for 3 days.

17. The method of claim 10 wherein the viral infection is an influenza viral infection and said prodrug has the structure:

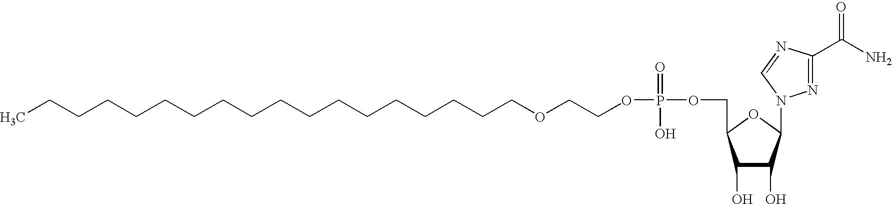

18. The method of claim 17 wherein the therapeutically effective amount is a dose between 100 and 4000 mg every 6 hours for 4 days, followed by a dose between 100 and 4000 mg every 8 hours for 3 days.

19. The method of claim 10 wherein the viral infection is a respiratory syndrome viral infection and said prodrug has the structure:

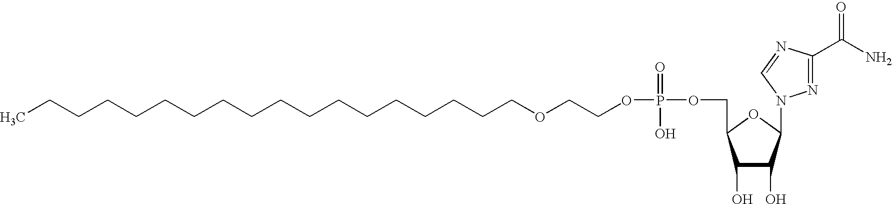

20. The method of claim 19 wherein the therapeutically effective amount is a dose between 100 and 4000 mg every 6 hours for 4 days, followed by a dose between 100 and 4000 mg every 8 hours for 3 days.
